(19) United States
(12) Patent Application Publication
(10) Pub. No.: US 2023/0118776 A1
(43) Pub. Date: Apr. 20, 2023

(54) Surgical Clamping Instrument

(71) Applicant: USB Medical, Ltd., Hatboro, PA (US)

(72) Inventors: John J. Sinisi, Warminster, PA (US); Jesse Sinisi, Warminster, PA (US); Stephen T. Epstein, Newtown, PA (US); Mattia Glauber, Milano (IT)

(21) Appl. No.: 17/506,611

(22) Filed: Oct. 20, 2021

(51) Int. Cl. A61B 17/29; A61B 17/3201

(52) U.S. Cl. CPC A61B 17/29; A61B 17/3201; A61B 2017/2808

(57) ABSTRACT

A clamping system having a clamp head, forceps, and a mechanical actuator. The clamp head has a first clamp or arm, a second clamp arm, a tubular neck, and a drive shaft that extends through the tubular neck. The first clamp arm is moved relative to the second clamp arm by selectively rotating the drive shaft. The forceps have a jaw section. The jaw section has a ring clamp that is sized to receive and retain the tubular neck on the clamp head. A mechanical actuator attaches to the forceps. The mechanical actuator has an elongated shaft that aligns with the drive shaft of the clamp head when the ring clamp is closed around the tubular neck of the clamp head. The alignment enables the mechanical actuator to engage the drive shaft and turn the drive shaft when the mechanical actuator is manipulated.

### BACKGROUND OF THE INVENTION

### 1. Field of the Invention

In general, the present invention relates to surgical clamping instruments. More particularly, the present invention relates to surgical clamping instruments that have clamp heads that detach from a handle inside the body. In this manner, a clamp head can be left within an incision while its handle is removed to make room for other surgical instruments.

### 2. Prior Art Description

There are many surgical procedures where a clamp is used deep within an incision. Traditional clamps have handles that are used to manipulate the clamp into position. The handles are also used to selectively open and close the clamp. The problem with such traditional clamps is that the handle of the clamp creates a physical barrier within the incision. The handle of the clamp can prevent a surgeon from viewing and accessing tissue of interest. The presence of the clamp handle also limits the room in which a surgeon can insert and manipulate other surgical instruments.

In the prior art, one solution to the clamp crowding problem involves utilizing a clamp with a clamp head that detaches from its handle. In this manner, the clamp head can be set in place using the handle, then the handle can be removed. The clamp head remains in place and the handle is no longer an obstruction. One such prior art clamping system is commonly referred to as a Glauber clamp and is exemplified by U.S. Pat. No. 8,409,229 to Wiedenbein.

There are certain disadvantages associated with prior art clamping systems, such as the Glauber clamp. One disadvantage is that once the clamp head is detached from the handle inside the body, it is often very difficult to reattach the handle to the clamp head inside the body. The clamp head and the handle must be specifically oriented before the clamp head and the handle can interconnect. Since the clamp head can, and often does, change orientations within the body, the clamp head rarely aligns with the handle when the handle is reinserted through an incision. The clamp head must then be manipulated into a better orientation using other surgical instruments. However, the presence of the other surgical instruments limits the room available to maneuver the clamp handle. Thus, the problem becomes self-complicating.

Another disadvantage of prior art Glauber-style clamping systems is that the clamps have large connection hubs. The connection hubs present large obstructions deep within surgical incisions. However, the connection hubs are required to receive the detachable handle and to transfer mechanical action from the handle to the clamp head to open and close the clamp head. If this connection cannot be made, the clamp must be surgically removed by other means. This adds significant complications to the surgical procedure.

A need therefore exists for an improved surgical clamping system that has a clamp head that detaches from a handle, wherein the parts can attach throughout a wide range of orientations. A need also exists for a detachable clamp head with a less bulky attachment hub and a detachable clamp to be easily opened and removed without the need of a specific detachable handle. These needs are met by the present invention as described and claimed below.

### SUMMARY OF THE INVENTION

The present invention is a clamping system for use during a surgical procedure. The clamping system includes a clamp head, forceps, and a mechanical actuator. The clamp head has a first clamp arm, a second clamp arm, a tubular neck, and a drive shaft that extends through the tubular neck. The first clamp arm is articulable and can be moved relative to the second clamp arm by selectively rotating the drive shaft. The drive shaft has a hub that can also be rotated with a standard forceps for ease of adjustment, added convenience, or emergency situations.

The forceps have a jaw section that can be manually opened and closed. The jaw section has a ring clamp thereon that opens and closes with the jaw section. The ring clamp is sized to receive and retain the tubular neck on the clamp head. The ring clamp can be closed around the neck across a wide range of approach angles. This is achieved in part by a unique side bevel on both the neck and the forceps allowing for an off-center approach with a self-centering connection.

A mechanical actuator is attached to the forceps. The mechanical actuator has an elongated shaft that aligns with the drive shaft of the clamp head when the ring clamp is closed around the tubular neck of the clamp head. The alignment enables the mechanical actuator to engage the drive shaft and turn the drive shaft when part of the mechanical actuator is manually turned.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is a perspective view of an exemplary embodiment of a clamp assembly with the clamp head attached to forceps;

FIG. 2 is a perspective view of the embodiment of FIG. 1 with the clamp head separated from the forceps;

FIG. 3 is an exploded view of the forceps of the exemplary embodiment;

FIG. 4 is an enlarged perspective view of the clamp assembly shown with a fragment of the forceps; and

FIG. 5 is a cross sectional view of the exemplary embodiment of FIG. 1 viewed along section line 5-5.

### DETAILED DESCRIPTION OF THE DRAWINGS

Although the present invention clamping system can be embodied in many ways, only one exemplary embodiment is illustrated. The exemplary embodiment is shown for the purposes of explanation and description. The exemplary embodiment is selected in order to set forth one of the best modes contemplated for the invention. The illustrated embodiment, however, is merely exemplary and should not be considered a limitation when interpreting the scope of the appended claims.

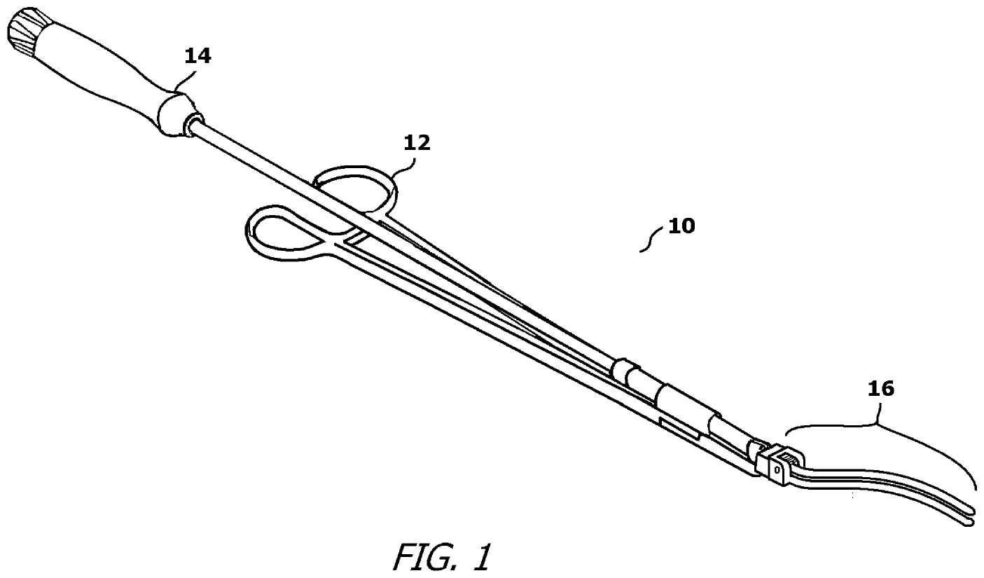

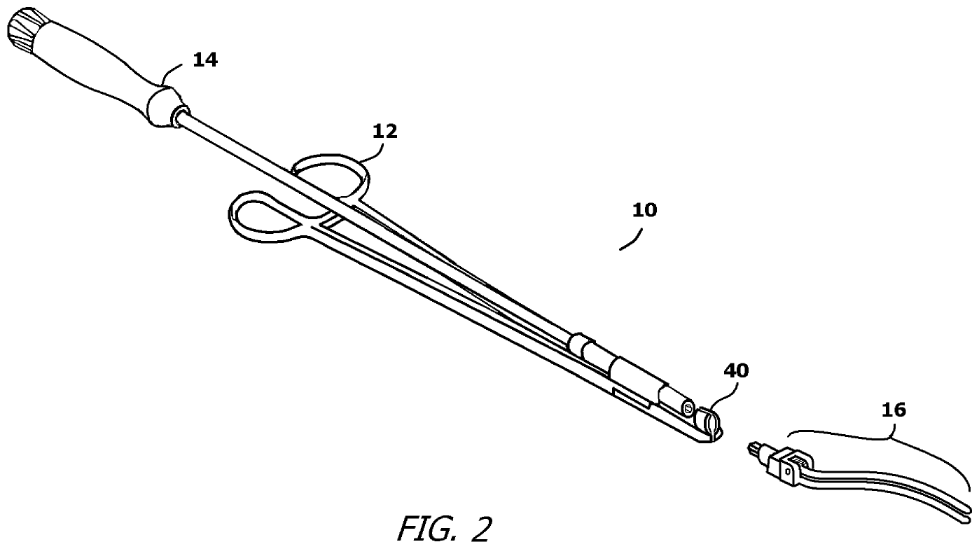

Referring to FIG. 1 and FIG. 2, a clamping system 10 is shown. The clamping system 10 includes a specialized forceps 12, a mechanical actuator 14, and a clamp head 16. The specialized forceps 12 supports the mechanical actuator 14. The specialized forceps 12 is also designed to selectively engage and release the clamp head 16. As such, the specialized forceps 12 is used to both insert the clamp head 16 into an incision and remove the clamp head 16 from an incision. The mechanical actuator 14 is used to selectively open and close the clamp head 16 when the clamp head 16 is engaged with the specialized forceps 12.

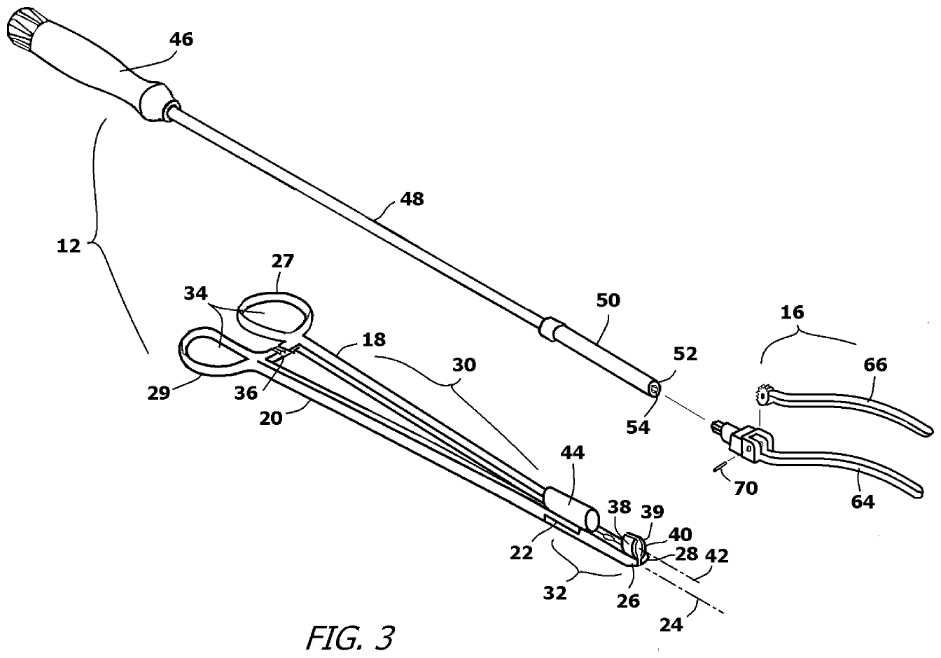

Referring to FIG. 3 in conjunction with FIG. 1 and FIG. 2, it can be seen that the specialized forceps 12 has two shanks 18, 20 that are joined together at a common scissor joint 22. The specialized forceps 12 is symmetrically disposed about a center line 24 that bisects the scissor joint 22. The first shank 18 has a first end 26 and an opposite second end 27. Likewise, the second shank 20 has a first end 28 and an opposite second end 29. The scissor joint 22 divides the specialized forceps 12 between a handle section 30 and a jaw section 32. The handle section 30 extends from the scissor joint 22 to the first ends 26, 28 of the shanks 18, 20. The first ends 26, 28 of the shanks 18, 20 terminate with finger loops 34. This allows the handle section 30 of the specialized forceps 12 to be gripped and manipulated in a traditional manner. The shanks 18, 20 can also include ratcheted extensions 36 that enable the two shanks 18, 20 to be selectively locked together at different angles.

The jaw section 32 of the specialized forceps 12 extends from the scissor joint 22 to the second ends 27, 29 of the two shanks 18, 20. At the second ends 27, 29 of the shanks 18, 20, each of the shanks 18, 20 terminates with a half ring 38, 39. Each half ring 38, 39 extends away from the shanks 18, 20 at a perpendicular to the bisecting centerline 24. When the jaw section 32 is closed and the shanks 18, 20 abut, the two half rings 38, 39 align to form a two-part ring clamp 40 with an internal radius R1. The two-part ring clamp 40 has a central axis 42 that is parallel to the bisecting centerline 24.

A guide tube 44 is mounted to the first shank 18 near the scissor joint 22. When the jaw section 32 is closed and the shanks 18, 20 abut, the guide tube 44 concentrically aligns with the two-part ring clamp 40, wherein both the guide tube 44 and the two-part ring clamp 40 share the same central axis 42.

The mechanical actuator 14 includes a handle 46. An elongated shaft 48 extends from the handle 46, wherein the elongated shaft 48 terminates at an engagement head 50. The engagement head 50 is cylindrical in shape and terminates at a distal end 52. A receptacle 54 is formed in the distal end 52 of the engagement head 50 that has a keyed shape, such as a hex shape or a spline shape. The purpose for the receptacle 54 is later explained. The engagement head 50 passes through, and is supported by, the guide tube 44 on the specialized forceps 12. The result is that the elongated shaft 48 is aligned with the central axis 42 of the guide tube 44. The mechanical actuator 14 is attached to the specialized forceps 12 in a manner where the elongated shaft 48 is still free to rotate about the central axis 42. The elongated shaft 48 can be selectively rotated by manually turning the handle 46.

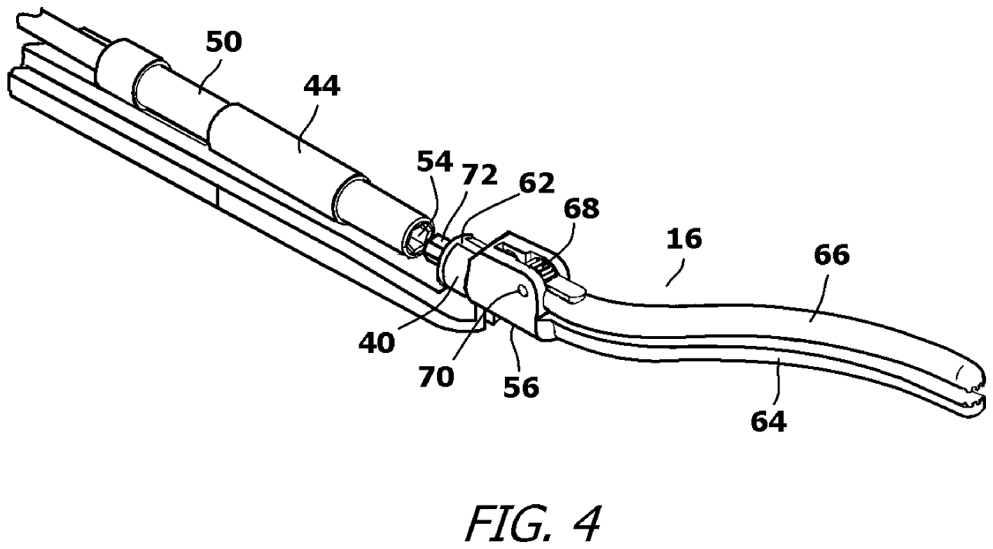

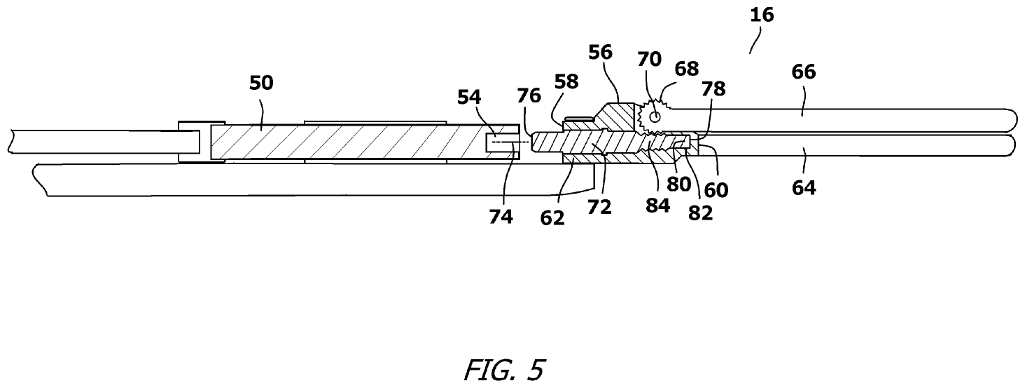

Referring to FIG. 4 and FIG. 5, in conjunction with FIG. 3, it can be seen that the clamp head 16 has a housing 56. The housing 56 has a first end 58 and an opposite second end 60. A tubular neck 62 is formed at the first end 58 of the housing 56. At the opposite second end 60 of the housing 56, two clamp arms 64, 66 extend from the housing 56. The clamp arms 64, 66 are mirrored in shape. The clamp arms 64, 66 include a base clamp arm 64 and an articulating clamp arm 66. The base clamp arm 64 is affixed to the second end 60 of the housing 56 and does not move relative to the housing 56. In operation, the base clamp arm 64 remains static. The articulating clamp arm 66 moves relative to the base clamp arm 64, therein causing the clamp head 16 to open and close.

The articulating clamp arm 66 has a pinion gear 68 formed at its base. Within the housing 56, a pivot pin 70 passes through the center of the pinion gear 68, wherein the pinion gear 68 is free to rotate about the pivot pin 70. The articulating clamp arm 66 rotates with the pinion gear 68. This selectively changes the angle of the articulating clamp arm 66 relative to the base clamp arm 64.

A drive shaft 72 extends into the housing 56 through the tubular neck 62 at the first end 58. The drive shaft 72 is elongated and has a long axis 74 that extends from a proximal end 76 to a distal end 78. The proximal end 76 of the drive shaft 72 has a keyed shape that mates with the shape of the receptacle 54 in the engagement head 50 of the elongated shaft 48. The drive shaft 48 has an axle nub 80 at its distal end 78. Within the housing 56, the axle nub 80 is received in a recess 82 within the housing 56. The drive shaft 72 is supported in the housing 56 both by the tubular neck 62 of the housing 56 and by the engagement of the axle nub 80 in the recess 82. Although supported by the housing 56, the drive shaft 72 is still free to rotate about its long axis 74.

A worm gear 84 is formed on the drive shaft 86 within the housing 56. The worm gear 84 intermeshes with the pinion gear 68 on the articulating clamp arm 66. As a consequence, when the drive shaft 72 rotates about its long axis 74, the worm gear 84 turns and rotates the pinion gear 68 and the articulating clamp arm 66 about the pivot pin 70.

Referring to all figures, it will be understood that to use the clamping system 10, the clamp head 16 is attached to the specialized forceps 12 and is advanced into an incision. The clamp head 16 is attached to the specialized forceps 12, by closing the jaw section 32 about the clamp head 16. More particularly, as the jaw section 32 is closed, the two-part ring clamp 40 closes over the tubular neck 62 on the clamp head 16. The engagement of the two-part ring clamp 40 around the tubular neck 62 aligns the tubular neck 62 and the drive shaft 86 with the elongated shaft 48 of the mechanical actuator 14. That is, the central axis 42 of the elongated shaft 48 and engagement head 50 is aligned with the long axis 74 of the drive shaft 86. Since the two-part ring clamp 40 is circular and the tubular neck 62 is circular, the two-part ring clamp 40 can engage the tubular neck 62 of the clamp head 16 from most any direction. Once the two-part ring clamp 40 is closed around the tubular neck 62, the two-part ring clamp 40 will automatically align the central axis 42 of the elongated shaft 48 with the long axis 74 of the drive shaft 72. This automatic alignment is of great benefit to a surgeon who need only grab the tubular neck 62 on the clamp head 16 with the two-part ring clamp 40 to achieve proper alignment.

Once the central axis 42 of the elongated shaft 48 and the engagement head 50 is aligned with the long axis 74 of the drive shaft 72, then the proximal end 76 of the drive shaft 72 is aligned with the receptacle 54 in the engagement head 50. The handle 46 can then be manually manipulated to advance the engagement head 50 toward the drive shaft 72. The keyed receptacle 54 on the engagement head 50 receives the keyed shape of the drive shaft 72 at its distal end 78. This creates a mechanical interconnection between the mechanical actuator 14 and the drive shaft 72. As a consequence, when the handle 46 of the mechanical actuator 14 is manually turned, the drive shaft 72 in the clamp head 16 will turn. As the drive shaft 72 turns, the worm gear 84 turns. The worm gear 84 turns the pinion gear 68. The rotation of the pinion gear 68 causes the articulating clamp arm to rotate about the pivot pin 70. This selectively opens and closes the clamp arms 64, 66 as directed by the manipulations of the handle 46 by a surgeon.

The clamp arms 64, 66 on the clamp head 16 can be adjusted as needed. Once the clamp head 16 is properly adjusted, the engagement head 50 can be retracted away from the drive shaft 72. The shanks 18, 20 of the specialized forceps 12 can be spread, whereby the clamp head 16 will detach from the specialized forceps 12. The specialized forceps 12 and the mechanical actuator 14 are removed from the incision, leaving the clamp head 16 within the incision. To retrieve the clamp head 16 from an incision, the process is reversed. The specialized forceps 12 is advanced into the incision. The two-part ring clamp 40 is closed around the tubular neck 62 of the mechanical actuator 14. This automatically aligns the drive shaft 72 of the clamp head 16 with the engagement head 50. The mechanical actuator 14 can then be advanced and turned to mechanically open the clamp head 16. The clamp head 16 can then be removed along with the specialized forceps 12 as a unit.

It will be understood that the embodiment of the present invention that is illustrated and described is merely exemplary and that a person skilled in the art can make many variations to that embodiment. For instance, the size and shape of the specialized forceps and the clamp head can be altered to meet the needs of a particular surgical procedure. All such embodiments are intended to be included within the scope of the present invention as defined by the claims.

What is claimed is:

1. A clamping system, comprising:
a clamp head having a first arm, a second arm, a tubular neck, and a drive shaft that extends through said tubular neck, wherein said first arm can be moved relative said second arm by rotating said drive shaft;
forceps having a jaw section with a ring clamp thereon, wherein said jaw section and said ring clamp open and close together, and wherein said ring clamp is sized to receive and retain said tubular neck on said clamp head; and
a mechanical actuator attached to said forceps, wherein said mechanical actuator has an elongated shaft that aligns with said drive shaft when said ring clamp on said forceps is closed around said tubular neck of said clamp head.

2. The clamping assembly according to claim 1, wherein said elongated shaft of said mechanical actuator extends along a central axis and said mechanical actuator includes a handle for selectively rotating said elongated shaft about said central axis and reciprocally moving said elongated shaft along said central axis.

3. The clamping assembly according to claim 2, wherein said elongated shaft terminates with an engagement head that moves with said elongated shaft, wherein said elongated shaft selectively interconnects with said drive shaft of said clamp head when said ring clamp on said forceps is closed around said tubular neck of said clamp head.

4. The clamping assembly according to claim 1, wherein said first arm rotates about a pivot pin.

5. The clamping assembly according to claim 4, wherein said first arm includes a pinion gear that rotates about said pivot pin.

6. The clamping assembly according to claim 5, wherein said clamp head includes a worm gear that is rotated by said drive shaft, wherein said worm gear turns said pinion gear and moves said first arm.

7. The clamping assembly according to claim 1, wherein said forceps includes a first shank and a second shank joined at a scissor joint, wherein said ring clamp includes a first half ring on said first shank and a second half ring on said second shank.

8. A clamping assembly, comprising:
a clamp head having a stationary arm, an articulable arm, a tubular neck, and a drive shaft that extends through said tubular neck and moves said articulable arm when turned;
forceps having a ring clamp that can be selectively opened and closed, and wherein said ring clamp is configured to receive and retain said tubular neck on said clamp head from multiple directions;
a mechanical actuator attached to said forceps, for engaging and turning said drive shaft when said ring clamp on said forceps is closed around said tubular neck of said clamp head.

9. The clamping assembly according to claim 8, wherein said mechanical actuator has an elongated shaft that extends along a central axis and a handle for selectively rotating said elongated shaft about said central axis and reciprocally moving said elongated shaft along said central axis.

10. The clamping assembly according to claim 8, wherein said elongated shaft terminates with an engagement head that moves with said elongated shaft, wherein said engagement shaft selectively interconnects with said drive shaft of said clamp head when said ring clamp is closed around said tubular neck of said clamp head.

11. The clamping assembly according to claim 8, wherein said articulable arm rotates about a pivot pin.

12. The clamping assembly according to claim 11, wherein said articulable arm includes a pinion gear that rotates about said pivot pin.

13. The clamping assembly according to claim 12, wherein said clamp head includes a worm gear that is rotated by said drive shaft, wherein said worm gear turns said pinion gear and moves said articulable arm.

14. The clamping assembly according to claim 8, wherein said forceps includes a first shank and a second shank joined at a common joint, wherein said ring clamp includes a first half ring on said first shank and a second half ring on said second shank.

15. A clamp head assembly, comprising:
a housing having a pivot pin and a tubular neck;
a stationary arm extending from said housing;
an articulable arm having gear teeth formed thereon, wherein said articulable arm is pivotably connected to said pivot pin in said housing; and
a drive shaft that extends through said tubular neck, wherein said drive shaft has a worm gear formed thereon that engages said gear teeth and rotates said articulable arm about said pivot pin when said drive shaft is turned.

16. The assembly according to claim 15, wherein said drive shaft has a first end that extends from said housing, wherein said first end has a keyed shape.
